(19) United States
(12) Patent Application Publication
(10) Pub. No.: US 2024/0150436 A1
(43) Pub. Date: May 9, 2024

(54) Application of MmPI in Preparation of Trypsin Inhibitors

(71) Applicant: SHAANXI UNIVERSITY OF TECHNOLOGY, Hanzhong (CN)

(72) Inventors: Youshan Li, Hanzhong (CN); Rui Zhu, Hanzhong (CN); Zhuxing Luo, Hanzhong (CN)

(73) Assignee: SHAANXI UNIVERSITY OF TECHNOLOGY, Hanzhong (CN)

(21) Appl. No.: 18/536,572

(22) Filed: Dec. 12, 2023

Related U.S. Application Data
(63) Continuation of application No. PCT/CN2023/111378, filed on Aug. 7, 2023.

(30) Foreign Application Priority Data
Sep. 16, 2022 (CN) CN202211127625.5

(51) Int. Cl. C07K 14/81

(52) U.S. Cl. CPC C07K 14/811

(57) ABSTRACT

The present disclosure relates to the field of genetic engineering or enzyme engineering, and in particular to an application of MmPI in preparation of trypsin inhibitors. The amino acid sequence of the MmPI is shown in SEQ ID NO.1. The present disclosure clarifies for the first time that MmPI in mulberry leaves has trypsin inhibitory activity and reveals its physical and chemical properties. The MmPI has good application prospects in preparing trypsin inhibitors. On the basis of knowing the physical and chemical properties of the MmPI, its activity may be accordingly eliminated, thereby it promotes the development and utilization of mulberry leaf resources in animal feed, provides new perspectives and ideas for the development and utilization of mulberry leaves in animal feed and health food, and enhances the economic benefits of mulberry resources.

### TECHNICAL FIELD

The present disclosure relates to the field of genetic engineering or enzyme engineering, and in particular to an application of MmPI in preparation of trypsin inhibitors.

### BACKGROUND

As a new and high-quality animal feed additive, mulberry leaves can not only improve the animal growth performance and the quality of poultry and livestock products, but also avoid the waste of mulberry leaf resources and improve the comprehensive economic benefits of the sericulture industry. However, due to the presence of anti-nutritional factors such as tannins, protease inhibitors and lectins in mulberry leaves, large amounts of mulberry leaves consumed by animals will seriously interfere with their metabolism and absorption of feed nutrients, thereby affecting the health of livestock and poultry and the yield and quality of livestock and poultry products, and greatly limiting the development and application of mulberry leaf resources in animal feed. Serine protease inhibitor (SPI) is a kind of protease inhibitors that is the most numerous and most intensively studied, it includes trypsin inhibitor (TI), chymotrypsin inhibitor (CI), elastase inhibitor (EI) and subtilisin inhibitor (SI), etc.

Based on the active site, mechanism of action of SPI, and its distribution in plants, plant SPI can be divided into eight families/categories, among which the Kunitz, Serpin, Bowman-Birk, PI-I and PI-II families have been studied more intensively. A total of 79 protease inhibitors (PIs), including 35 SPIs that belong to the Kunitz, Serpin and PI-I families, were identified in the Morus notabilis genome. After analyzing the expression of different SPI family genes in various tissues, it was found that 8 Kunitz-family SPI genes and 1 Serpin-family SPI gene were mainly expressed in mulberry leaves. It is found by Western Blot detection that the serine protease inhibitor MmKPI-9 is expressed in mulberry leaves, but TI activity bands are not detected by in-gel activity staining. Wang Dandan used in-gel activity staining technology to detect multiple CI activity bands in the white latex flowing out of the petioles of mulberry leaves, but no CI activity was detected in the mulberry leaves. At present, there are few research reports on anti-nutritional factor SPIs in mulberry leaves, and the sequence information, activity, and physical and chemical properties of these SPIs are still unclear.

### SUMMARY

In order to solve the above technical problems, the present disclosure provides an application of MmPI in preparing trypsin inhibitors, wherein an amino acid sequence of the MmPI is shown in SEQ ID NO. 1.

Based on the same inventive concept, the present disclosure also provides an isolated gene fragment, wherein a nucleotide sequence of the gene fragment is shown in SEQ ID NO. 2.

Based on the same inventive concept, the present disclosure also provides a plasmid carrying the gene fragment.

Based on the same inventive concept, the present disclosure also provides a host expression strain carrying the plasmid.

Based on the same inventive concept, the present disclosure also provides an application of an expression product of the strain in preparing trypsin inhibitors, wherein the expression product is MmPI with an amino acid sequence as shown in SEQ ID NO.1.

Based on the same inventive concept, the present disclosure also provides an activity elimination method of the MmPI, comprising: placing the MmPI in an environment of 121° C. and 0.21 MPa for 20 minutes; or, eliminating the MmPI by Maillard reaction mediated by reducing sugar.

The disclosure has the following beneficial effects:

The present disclosure clarifies for the first time that MmPI in mulberry leaves has trypsin inhibitory activity and reveals its physical and chemical properties. The MmPI has good application prospects in preparing trypsin inhibitors. On the basis of knowing the physical and chemical properties of the MmPI, MmPI activity may be also be eliminated, thereby promoting the development and utilization of mulberry leaf resources in animal feed, providing new perspectives and ideas for the development and utilization of mulberry leaves in animal feed and health food, and enhancing the economic benefits of mulberry resources.

### BRIEF DESCRIPTION OF THE DRAWINGS

FIG. 1 shows the electrophoretic detection of the PCR (Polymerase Chain Reaction) product of MmPI_{(Ma)} in (A), and the electrophoretic detection of the bacterial liquid PCR product of MmPI_{(Ma)} in (B).

FIG. 2 shows the nucleotide sequence of MmPI and its derived amino acid sequence. The gray background part is the signal peptide sequence, and the underlined part is the PI domain. The start codon (ATG) and stop codon (TAA) are shown in the boxes.

FIG. 3 shows SDS-PAGE analysis of MmSPI6 and MmPI expressed in BL21(DE3) cells in (A), and SDS-PAGE analysis of MmSPI6 and MmPI expressed in Origami 2(DE3) cells in (B). “S” indicates soluble protein; “U” indicates unsoluble protein; “Control”, cell lysate of BL21(DE3) and Origami 2(DE3) strains transformed into p28 empty vector, MmSPI6 is another serine protease inhibitor.

FIG. 4 shows the TI (A) and CI (B) activity analysis of MmPI expressed in BL21(DE3) cells. “TI” and “CI” stand for trypsin inhibitor and chymotrypsin inhibitor, respectively. Bombyx mori hemolymph from day-5 fifth-instar larvae was used as a positive control. “Control”, cell lysate of BL21(DE3) strain transformed into p28 empty vector. The arrow indicates protease inhibitor activity bands.

FIG. 5 shows the TI (A) and CI (B) activity analysis of MmPI expressed in Origami 2(DE3) cells. “TI” and “CI” stand for trypsin inhibitor and chymotrypsin inhibitor, respectively. Bombyx mori hemolymph from day-5 fifth-instar larvae was used as a positive control. “Control”, cell lysate of Origami 2(DE3) strain transformed into p28 empty vector. The arrow indicates protease inhibitor activity bands.

FIG. 6 shows the effect of different pH on the activity of MmPI.

FIG. 7 shows the effect of high temperature and high pressure on the activity of MmPI.

FIG. 8 shows the effect of β-mercaptoethanol on the activity of MmPI.

FIG. 9 shows the effect of Maillard reaction on the activity of MmPI.

### DETAILED DESCRIPTION OF EMBODIMENTS

The present disclosure will be described in detail below with reference to the accompanying drawings and specific embodiments, but it should not be understood as limitations of the present disclosure. Unless otherwise specified, the technical solutions used in the following embodiments or examples are optional. The materials, reagents, etc. used in the following embodiments or examples can all be obtained from commercial sources, unless otherwise specified.

The following embodiments involve p28 expression vectors deposited by the Institute of Vitamin D Physiology and Applications, Shaanxi University of Science and Technology.

### Embodiment I

1 Experimental method

1.1 RNA extraction from “Jin 10” leaves and synthesis of first-strand of cDNA

1.1.1 Extraction of mulberry leaves RNA

Use Eastep Super total RNA extraction kit (Promega Company) and operate according to the instructions.

1.1.2 Synthesis of the first strand of cDNA

Denature and melt the total RNA. Take 4μg of total RNA, add 1 μL of Oligo(dT), and make up to 10 μL with RNase-free water. Place them in the PCR amplifier at 42° C. for 30min, and then at 85° C. for 5 s. After the reaction is completed, quickly take them out and place them in ice, dilute them 2.5 times and store them at −20° C.

RT-PCR reaction system:

| | | |
|---|---|---|
| 2 × ES | 10 | μL |
| RNase-free water | 3.4 | μL |
| Oligo(dT) | 1 | μL |
| gDNA Remover | 1 | μL |
| Easy mix | 1 | μL |
| RNA | 3.6 | μL |

1.2 Construction of expression vector

1.2.1 PCR amplification of target fragments of inhibitors in mulberry leaves

Based on the CDS sequence of protease inhibitors in Morus alba, primers were designed for the MmPI gene (the names were designed and named by the applicant's team). The primer sequences are shown in Table 1.

TABLE 1
Primers used for cloning of
protease inhibitor MmPI

| | Sequence of primers | SEQ ID |
|---|---|---|
| Primers | (5′ to 3′) | NO. |
| MmPI_{(Ma)}-F | CGCCATATGATGGCTAT | 3 |
| | CTCCAACCAAGAA | |
| MmPI_{(Ma)}-R | ATTTGCGGCCGCTTAAC | 4 |
| | CAACACGAGGAACTTCA | |

Note:
The underline indicates the restriction enzyme cutting site.
Nde I site: CATATG; Not I site: GCGGCCGC.
The subscript “Ma” indicates the primers designed based on the database of Morus alba.

Use Jin 10 leaf cDNA as a template for PCR amplification. The PCR system is as

| | | |
|---|---|---|
| Forward Primer | 0.5 | μL |
| Reverse Primer | 0.5 | μL |
| 5 × FastPfu Fly buffer | 5 | μL |
| 2.5 mM dNTPs | 2 | μL |
| ddH₂O | 9.5 | μL |
| cDNA | 1 | μL |
| 50 mM MgSO₄ | 1 | μL |
| 5 × PCR stimulant | 5 | μL |
| FastPfu Fly DNA Polymerase | 0.5 | μL |
| Total | 25 | μL |

PCR amplification program: pre-denaturation at 95° C. for 2 min; 35 cycles of denaturation at 95° C. for 30 s, annealing at 55° C. for 30 s, and extension at 72° C. for 30 s; and extension at 72° C. for 10 min. The PCR product was separated by 1.5% agarose gel electrophoresis and purified with reference to the EasyPure PCR Purification Kit. The PCR product and p28 vector were double-enzyme digested. The double-enzyme digestion reaction system is shown in Table 2.

TABLE 2
Double-enzyme digestion reaction system

| | PCR product double- | | p28 vector double-enzyme | |
|---|---|---|---|---|
| Reagents | enzyme digestion system | | digestion system | |
| PCR product | 25 | μL | | |
| p28 plasmid | | | 20 | μL |
| 10 × H buffer | 5 | μL | 5 | μL |
| BSA(0.1%) | 5 | μL | 5 | μL |
| Nde I | 2 | μL | 2.5 | μL |
| Not I | 2 | μL | 2.5 | μL |
| ddH₂O | 11 | μL | 15 | μL |
| Total volume | 50 | μL | 50 | μL |

After digestion at 37° C. overnight, add 10x Loading buffer to terminate digestion. The digested products were separated by 1.5% agarose gel electrophoresis and gel-extracted with reference to the EasyPure Quick Gel Extraction Kit.

1.2.2 Ligate the target fragment to the p28 vector

Ligate the target fragment to the p28 vector at 16° C. for 2 hours. The ligation system is as follows:

| | |
|---|---|
| Target fragment | 1 μL |
| p28 vector | 6 μL |
| T4 DNA ligase buffer | 2 μL |
| T4 DNA ligase | 1 μL |

1.2.3 Transformation

Transform the ligation product into Escherichia coli DH5α competent cells. The specific transformation steps are as follows:

1) Place the DH5α competent cells on ice. When they have just melted, add the ligation product, mix gently by pipetting, and let stand on ice for 30 minutes.

2) Heat shock at 42° C. for 90 seconds, then quickly place them on ice and let stand for 5 minutes.

3) Add 900 μL of 2-YT liquid medium without antibiotics and incubate at 37° C. and 220 rpm for 1 hour.

4) Centrifuge at 3500 rpm for 5 minutes, discard 800 μL of the supernatant, resuspend the pellet and supernatant with a pipette, add resuspension liquid to 2-YT solid medium containing kanamycin resistance, and use a sterilized coating stick to lightly coat evenly.

5) Place it upright for 10 minutes in a 37° C. constant-temperature incubator, then invert it for 12 hours.

1.2.4 Bacterial liquid PCR screening of positive clones and sequencing

Pick 6 large, round white single colonies, inoculate them into 600 μL of 2-YT liquid medium containing kanamycin resistance, and culture them with shaking at 37° C. and 220 rpm for 4 hours. Take 2 μL of bacterial liquid as a template for bacterial liquid PCR. The bacterial liquid PCR reaction system is as follows:

| | | |
|---|---|---|
| Forward Primer | 0.5 | μL |
| Reverse Primer | 0.5 | μL |
| 2 × Fine Taq mix | 12.5 | μL |
| Bacterial liquid | 2 | μL |

The PCR program was: pre-denaturation at 95° C. for 5 min; 30 cycles of denaturation at 95° C. for 30 s, annealing at 55° C. for 30 s, and extension at 72° C. for 1 min or 75 s; and extension at 72° C. for 10 min. The PCR product was tested by 1.5% agarose gel, and the bacterial liquid that could amplify the target band was a positive clone. Select three positive clones with bright bands, take 200 μL of each and send them to Sangon Biotech (Shanghai, China) for sequencing.

1.2.5 Preparation of glycerol bacteria and plasmid extraction

According to the ratio of 1/100˜1/1000, take the correctly sequenced bacterial liquid and put it into the 2-YT liquid medium containing kanamycin resistance, shake-culture at 37° C. and 220 rpm for 12 hours; take 300 μL at of bacterial liquid and mix with 200 μL at of 50% glycerol to prepare glycerol bacteria and store it at −20° C. Pipette 2 mL of bacterial liquid and extract the plasmid with reference to the EasyPure Plasmid MiniPrep Kit.

1.3 Prokaryotic expression of MmPI

1.3.1 Transform into Escherichia coli expression strain

Transform the plasmid into Escherichia coli BL21(DE3) and Origami 2(DE3) strains. The transformation steps are as follows:

1) Place the BL21(DE3) or Origami 2(DE3) competent cells stored at −80° C. on ice. When they have just melted, take 1 μL of plasmid and slowly add it to the competent cells and mix gently, bath for 30 minutes.

2) Heat shock at 42° C. for 90 seconds. After the heat shock is completed, quickly insert it into ice and cool it for 5 minutes.

3) Add 900 μL of 2-YT liquid medium without antibiotics and incubate at 37° C. and 220 rpm for 1 hour.

4) Centrifuge at 3500 rpm for 5 minutes and discard 800 μL of supernatant.

5) Mix the remaining supernatant and pellet with a pipette, and place the suspension in a 2-YT solid medium containing one antibiotic (kanamycin resistance) or three antibiotics (kanamycin, streptomycin, and tetracycline), use a sterilized coating stick to gently spread evenly.

6) Place the solid plate upright in a 37° C. incubator for 10 minutes, then invert it for 12 hours.

1.3.2 Induced expression

1) Pick large and round single colonies and culture them in 600 μL of 2-YT liquid culture medium containing one antibiotic (kanamycin resistance) or three antibiotics (kanamycin, streptomycin, tetracycline), incubate overnight at 37° C. and 220 rpm on a constant temperature shaker.

2) Take 150 μL of bacterial liquid that was shaken overnight in 15 mL of 2-YT liquid culture medium with the corresponding antibiotics, and culture it at 37° C. and 220 rpm until the bacterial liquid OD₆₀₀=0.6˜1.0, then quickly insert it into ice to slow down growth.

3) Add 0.1 M IPTG stock solution (i.e. working concentration is 0.2 mM) at a ratio of 1/500, and induce at 37° C. and 220 rpm for 5 h or 16° C. and 220 rpm for 20h.

4) After induction, centrifuge at 4° C. and 6000 rpm for 20 minutes and discard the supernatant.

5) Add 1.5 mL of 1× binding buffer to resuspend the bacterial cells, centrifuge at 4° C. and 6000 rpm for 10 min, and discard the supernatant.

6) Add 1 mL of 1× binding buffer to resuspend the bacterial cells, centrifuge again, and discard the supernatant.

7) Finally add 450 μL of 1× binding buffer to resuspend the bacterial cells and store at −20° C.

1.3.3 Cell disruption

Place the bacterial cell mixture in the ice-water mixture, use a 30W ultrasonic disruptor to crush the bacterial cell mixture until the bacterial cell mixture is translucent, centrifuge at 4° C. and 16000 g for 30 minutes, separate the supernatant, and add 250 μL of 1× binding buffer to the sediment for resuspending to obtain an inclusion body solution, and store the supernatant and precipitate at −20° C.

1.4 SDS-PAGE and Native PAGE, and Activity staining of MmPI

1.5 Effects of different pH, high temperature and high pressure, reducing agent and Maillard reaction on the activity of MmPI 2 Results and analysis

2.1 Construction of MmPI expression vector

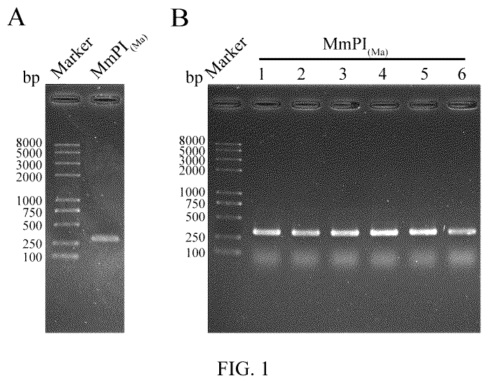

PCR amplification of MmPI(Ma) resulted in a bright and single band (FIG. 1A). The PCR product was connected to the p28 vector and transferred into DH5α competent cells, the bacterial liquid PCR was performed, and 1% agarose gel electrophoresis was used to implement detection, the results are shown in FIG. 1B. The bacterial liquid of positive clones was selected, and sequencing confirmed that MmPI_{(Ma)} was successfully cloned. MmPI₍ₘₐ₎ is named as: MmPI. Next, we will extract gene plasmid and perform prokaryotic expression of it.

2.2 Primary structure analysis of MmPI

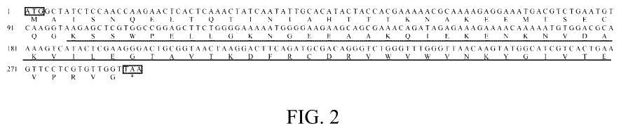

The CDS coding frame of MmPI consists of 288 nucleotides, the sequence is shown in SEQ ID NO.2. Encode a protein of 95 amino acids, the sequence is shown in SEQ ID NO.1. This protein has no signal peptide (FIG. 2). The mature form of MmPI protein has a molecular weight of 10636.04 Da, an isoelectric point (pI) of 5.84, and has a PI domain.

2.3 Prokaryotic expression of MmPI

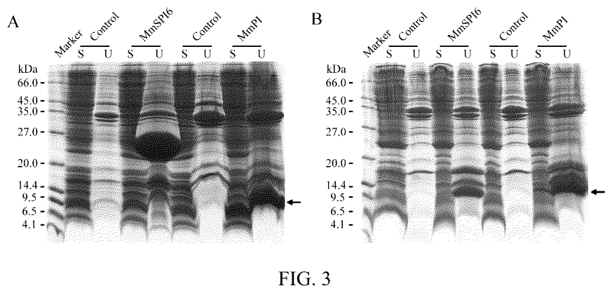

In order to achieve prokaryotic expression of the target gene, the plasmid was transferred into two expression strains of Escherichia coli BL21(DE3) and Origami 2(DE3), and IPTG with a working concentration of 0.2 mM was used to induce expression. The target protein was separated and detected using 16.5% SDS-PAGE. The results showed that MmPI was expressed in the supernatant of both BL21(DE3) and Origami 2(DE3) strains (FIG. 3).

2.4 Activity analysis of MmPI

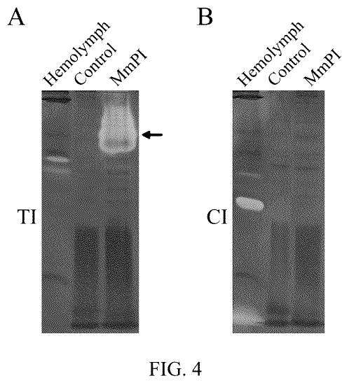

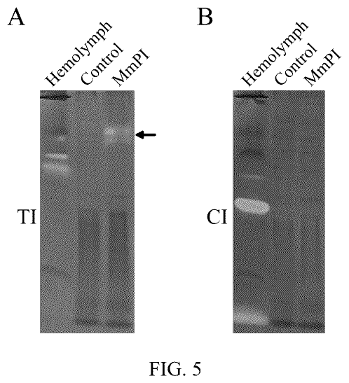

In order to analyze the inhibitory activity of MmPI protein against trypsin and chymotrypsin, we used 10% Native PAGE to separate the induced expression of MmPI protein in BL21(DE3) and Origami 2(DE3) strains, and then performed in-gel activity staining. The results showed that MmPI induce-expressed in BL21(DE3) (FIG. 4) and Origami 2(DE3) (FIG. 5) strains could strongly inhibit trypsin activity, but did not inhibit chymotrypsin activity. Compared with the Origami 2(DE3) strain, the MmPI protein in BL21(DE3) is more active.

2.5 Effects of pH, temperature, reducing agent and Maillard reaction on MmPI activity

(1) Effect of different pH on MmPI activity

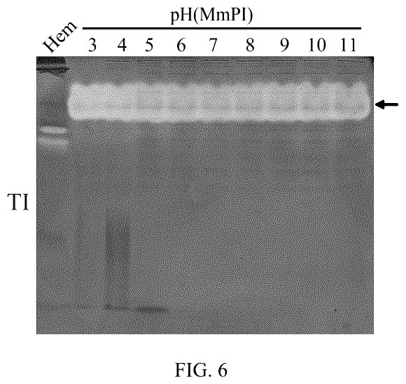

As shown in FIG. 6, the protease inhibitor MmPI has strong acid-base stability, and its inhibitory activity against trypsin remains basically stable within the pH range of 3 to 11.

(2) Effect of high temperature and high pressure on MmPI activity

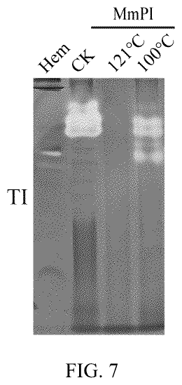

In order to verify the effect of high temperature or combination of high temperature and high pressure on the activity of protease inhibitor MmPI, we treated the protease inhibitor at 121° C. and 0.21MPa or 100° C. for 20 min, and then analyzed its inhibitory activity against trypsin by Native PAGE and in-gel activity staining. The results of in-gel activity staining (FIG. 7) show that compared with the control group, treatment at 100° C. will slightly reduce the inhibitory activity of MmPI against trypsin, while the combination of high temperature and high pressure (121° C. and 0.21 MPa) can make the inhibitory activity of MmPI against trypsin completely lost. It should be noted that the position of the MmPI active band migrated downward after heat treatment for 20 minutes, suggesting that heating caused a change in the conformation of MmPI. In short, the combination of high temperature and high pressure can basically eliminate the inhibitory activity of MmPI on trypsin.

(3) Effect of reducing agents on MmPI activity

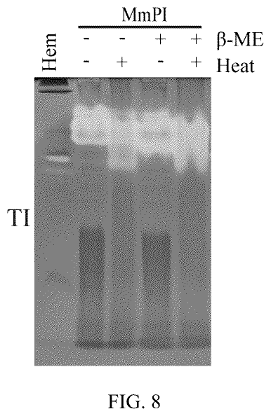

In order to explore the effect of reducing agents on the activity of MmPI, we used β-mercaptoethanol to treat the protease inhibitor MmPI and analyzed its inhibitory activity against trypsin. The results are shown in FIG. 8, regardless of the presence or absence of β-mercaptoethanol, heating treatment has no obvious effect on the activity of MmPI. However, heating can cause the active band of MmPI to migrate downward, again suggesting that heating will cause its conformation to change. In summary, β-mercaptoethanol has no significant effect on the inhibitory activity of MmPI.

(4) Effect of glucose-mediated Maillard reaction on MmPI activity

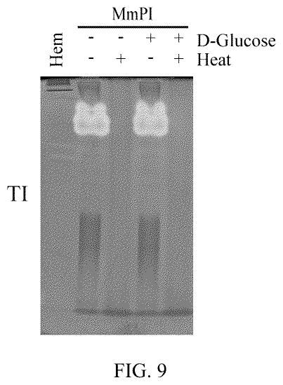

As shown in FIG. 9, heating at 100° C. for 60 minutes basically lost the inhibitory activity of MmPI; when glucose was present without heating, MmPI had no significant effect on the inhibitory activity of the protease. MmPI is completely inactivated in the presence of glucose and heating. The above results indicate that the Maillard reaction mediated by glucose can reduce the activity of the MmPI.

Although the preferred embodiments of the present disclosure have been described, additional changes and modifications to these embodiments may be made by those skilled in the art once the basic inventive concepts are apparent. Therefore, it is intended that the appended claims should be construed to include the preferred embodiments and all changes and modifications that fall within the scope of the present disclosure.

Obviously, various changes and modifications to the present disclosure may be made by those skilled in the art without departing from the spirit and scope of the present disclosure. Thus, if these changes and modifications of the present disclosure fall within the scope of the claims of the present disclosure and equivalent technologies thereof, the present disclosure is also intended to include these modifications and variations.

### CROSS-REFERENCE TO SEQUENCE LISTING XML FILE

This application contains a Sequence Listing XML as a separate part of the disclosure, which presents nucleotide and/or amino acid sequences and associated information using the symbols and format in accordance with the requirements of 37 CFR-1.831-1.835. The XML file named “MmPI Sequences.xml”, created Dec. 10, 2023, 4,829 bytes in size, is submitted herewith and is incorporated by reference in its entirety.

What is claimed is:

1. A method of preparing trypsin inhibitors, comprising adding an effective amount of an MmPI comprising an amino acid sequence of the MmPI shown in SEQ ID NO. 1 to a preparation of protease inhibitors.

2. An isolated gene fragment, wherein a nucleotide sequence of the gene fragment is shown in SEQ ID NO. 2.

3. A plasmid carrying the gene fragment of claim 2.

4. A host expression strain carrying the plasmid of claim 3.

5. A method of expressing a product of the strain of claim 4, wherein the expression product is MmPI with an amino acid sequence as shown in SEQ ID NO.1.

6. A method of eliminating activity of the MmPI of claim 1, comprising: placing the MmPI in an environment of 121° C. and 0.21 MPa for 20 minutes; or, eliminating the MmPI by Maillard reaction mediated by reducing sugar.


SEQUENCE LISTING (4 sequences)

SEQ ID NO: 1 - protein, 95 aa, Morus alba
MAISNQELTQTINIAHTTTKNAKEEMTSECQGKSSWPELLGKNGEEAAKQIEKENKNVDAKVILEGTAVTKDFRCDRVWVWVNKYGIVTEVPRVG

SEQ ID NO: 2 - DNA, 288 bp, Morus alba
  source 1..288: mol_type = genomic DNA
atggctatctccaaccaagaactcactcaaactatcaatattgcacatactaccacgaaaaacgcaaaagaggaaatgacgtctgaatgtcaaggtaagagctcgtggccggagcttctggggaaaaatggggaagaagcagcgaaacagatagagaaagaaaacaaaaatgtggacgcaaaagtcatactcgaagggactgcggtaactaaggacttcagatgcgacagggtctgggtttgggttaacaagtatggcatcgtcactgaagttcctcgtgttggttaa

SEQ ID NO: 3 - DNA, 30 bp, synthetic construct
  source 1..30: mol_type = other DNA
cgccatatgatggctatctccaaccaagaa

SEQ ID NO: 4 - DNA, 34 bp, synthetic construct
  source 1..34: mol_type = other DNA
atttgcggccgcttaaccaacacgaggaacttca